(19) United States
(12) Patent Application Publication
(10) Pub. No.: US 2008/0156674 A1
(43) Pub. Date: Jul. 3, 2008

(54) APPARATUSES AND SYSTEMS FOR STORING, DISPENSING, AND RECONSTITUTING MATERIALS

(75) Inventors: David Correale, West Chester, PA (US); Michael Fischer, Jenkintown, PA (US); Harold Clupper, West Chester, PA (US)

Correspondence Address: KENYON & KENYON LLP, 1500 K STREET N.W., SUITE 700, WASHINGTON, DC, 20005, US

(73) Assignee: PURICORE, INC., Malvern, PA (US)

(21) Appl. No.: 11/937,352

(22) Filed: Nov. 8, 2007

Related U.S. Application Data
(60) Provisional application No. 60/857,785, filed on Nov. 9, 2006.

(51) Int. Cl. B65D 25/08; A61B 19/00

(52) U.S. Cl. 206/221; 604/416

(57) ABSTRACT

The present invention is directed to apparatuses and systems for storing, dispensing, and reconstituting materials. Apparatuses may comprise a container to store a material having an open end and a dispensing aperture. A cover may be configured to seal the open end of the container and a sleeve can include a passageway to slidably receive the container. The container may be moveable within the sleeve between a storing position wherein the dispensing aperture is covered and a dispensing position wherein the dispensing aperture is uncovered to dispense the material. Systems may additionally comprise a reservoir having an opening and the sleeve configured for mounting on the reservoir opening so that when the container is moved to the dispensing position the material is dispensed into the reservoir to form a reconstituted material.

### CROSS-REFERENCE TO RELATED APPLICATIONS

This application claims the benefit of U.S. Provisional Application No. 60/857,785 under 35 U.S.C. 119(e), filed on Nov. 9, 2006, the entire contents of which are hereby incorporated by reference.

### TECHNICAL FIELD

The present invention generally relates to apparatuses and systems for storing, dispensing, and reconstituting materials.

### BACKGROUND

Sterilizing, disinfecting, or biocidal solutions may be used on surfaces for microbial action against spores, viruses, fungi, and bacteria. Such solutions can remove biofilms and may also eliminate further growth. These solutions have a broad range of applications in both the medical and non-medical environments. For example, these solutions may be used for the preservation of poultry and fish, general agricultural and petrochemical uses, the breaking down of biofilm, water treatment, general disinfection in medical and veterinary applications, and any other application in which there may be a desire to free a surface of living organisms.

Conventional biocidal solutions include formaldehyde, peracetic acid, and glutaraldehyde. Conventional solutions may have sensitizing properties to the handler, irritate the skin, and can be harmful to humans. These conventional solutions have limited applications due to their toxic nature and handling precautions.

In order to address these drawbacks, biocidal solutions produced by the electrolysis of saline may be used. One such electrolyzed saline solution is produced by the applicant under the trademark STERILOX® (PuriCore, Inc., Lapp, PA).

Electrolyzed saline solutions produced by electrolysis can be generated by passing an electrolyte, such as saline solution, through an electrolytic cell comprising an anode chamber, a cathode chamber, and a separator, such as the electrolytic cell described in UK Patent Nos. 2,253,860, 2,274,113, and 2,253,860, which are hereby incorporated by reference in their entirety. Electrolyzed saline solutions can be generated in small volumes as described in GB 2393737, the entire contents of which is also hereby incorporated by reference. One problem with generating electrolyzed saline solutions, is that the electrolyte needed to prepare such solutions are difficult and costly to ship to the end user, given the size and volume of the container carrying such an electrolyte. Accordingly, there is a need in the art for a more cost-effective and flexible way to deliver an electrolyte or other material that allows the end user to reconstitute the electrolyte or other material at the point of use.

### BRIEF DESCRIPTION

The present invention is directed to apparatuses and systems for storing, dispensing, and reconstituting materials at the point of use.

In accordance with embodiments of the present invention, an apparatus may comprise a container to store a material having an open end and a dispensing aperture. A cover may be configured to seal the open end of the container and a sleeve can include a passageway to slidably receive the container. The container may be moveable within the sleeve between a storing position wherein the dispensing aperture is covered and a dispensing position wherein the dispensing aperture is uncovered to dispense the material.

Embodiments of the invention may also include systems for storing, dispensing, and reconstituting materials. A system in accordance with the invention may comprise an apparatus including a container to store a material having an open end and a dispensing aperture. A cover may be configured to seal the open end of the container and a sleeve can include a passageway to slidably receive the container. The container may be moveable within the sleeve between a storing position wherein the dispensing aperture is covered and a dispensing position wherein the dispensing aperture is uncovered to dispense the material. A reservoir(s) containing a substance in which the material is dissolved or otherwise reconstituted may also be provided having an opening. The sleeve of the apparatus may be configured for mounting on the reservoir opening so that when the container is moved to the dispensing position the material is dispensed into the reservoir to form a reconstituted material. In certain embodiments, the reservoir is a liquid reservoir and the reconstituted material is a solution.

The invention may be embodied by numerous apparatuses and systems, and the description and drawings provided herein are examples of the invention. Other embodiments, which incorporate some or all of the features, are also possible.

### BRIEF DESCRIPTION OF THE DRAWINGS

Referring to the drawings, which form a part of this disclosure:

FIG. 1 shows an exploded view of an apparatus for storing and dispensing material as may be employed in accordance with embodiments of the present invention;

FIG. 2 shows the container and cover of the apparatus of FIG. 1 as may be employed with embodiments of the present invention;

FIGS. 3a-b show the split ring of FIG. 1 as may be employed in accordance with embodiments of the present invention;

FIG. 4a shows a sleeve of the apparatus of FIG. 1 and FIG. 4b shows alignment tabs of the sleeve located within dispensing apertures of the container as may be employed in accordance with embodiments of the present invention;

FIG. 4c illustrates a second cover and FIG. 4d illustrates the second cover sealing the bottom of the apparatus of FIG. 1 in a storing position as may be employed in accordance with embodiments of the present invention;

FIG. 5a is an exploded view of a bottom cap and a container to be fitted in the bottom receptacle. FIG. 5c illustrates the container of FIG. 5a fitted in the bottom receptacle.

FIGS. 6a-b show the apparatus of FIG. 1 in the storing and dispensing positions;

FIGS. 7a-d show a system for using the apparatus of FIG. 1 with a reservoir as may be employed in accordance with embodiments of the present invention; and

FIG. 8 is a flow chart of method steps that may be employed in accordance with embodiments of the present invention.

### DETAILED DESCRIPTION

The present invention generally relates to apparatuses, kits and systems and methods which may be used to store, dispense, and reconstitute materials into solutions or another reconstituted forms at the point of use.

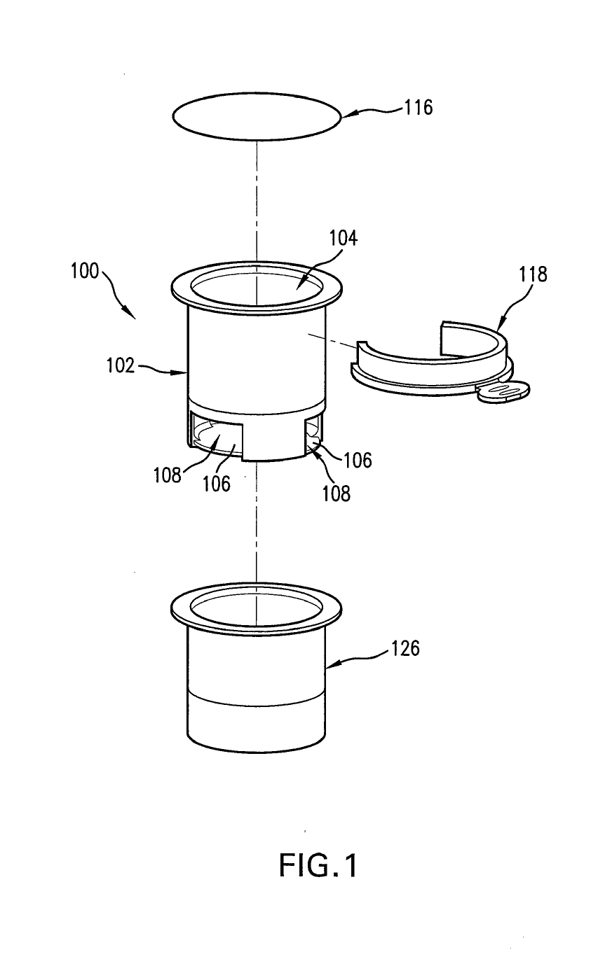

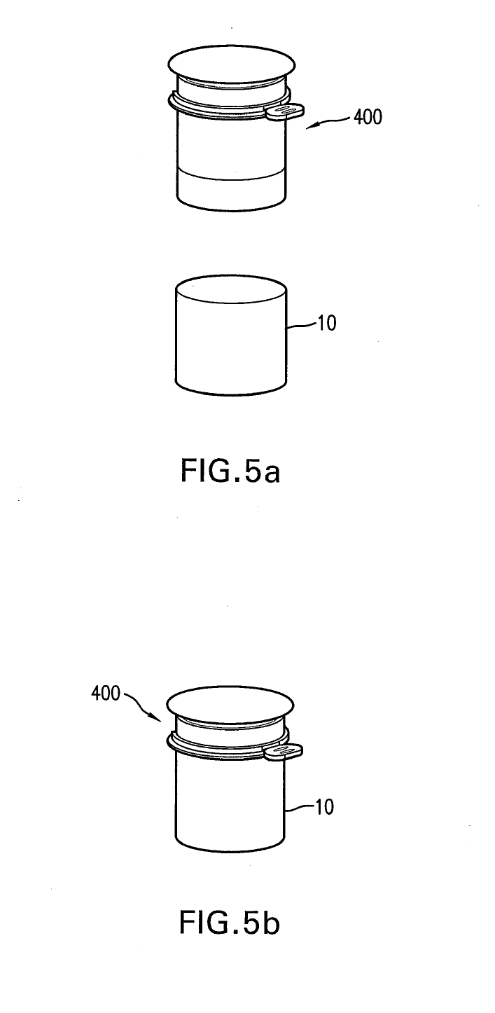

FIG. 1 illustrates an embodiment of an apparatus 100 for storing and dispensing materials. The apparatus 100 includes a container 102 configured to store the materials. The container 102 may have open and closed ends 104, 106 and one or more dispensing aperture(s) 108. The apparatus may also include a cover 116, a slip ring 118, and a sleeve 126. The container 102 may be moveable within the sleeve 126 between a storing position (FIG. 5a) where the dispensing aperture(s) 108 are covered and a dispensing position (FIG. 5b) where the dispensing aperture(s) 108 are uncovered to dispense the materials stored within the container 102.

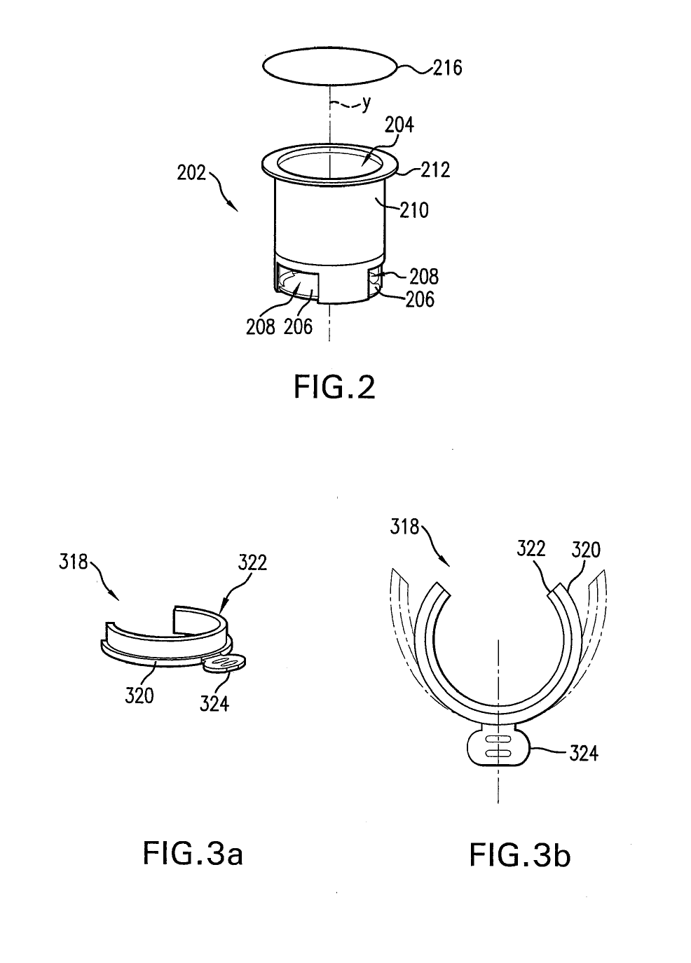

Referring to FIG. 2, a container 202 is shown which may be used in accordance with an embodiment of the present invention to store and dispense materials. As seen in the example, the container 202 may have a generally tubular body 210 including open and closed ends 204, 206. A flange 212 may extend outwardly from and perpendicularly to a longitudinal axis (y) of the container 202. In this example, the flange 212 may be located proximate to the open end 204 of the container 202.

Also in this example, it can be seen that the dispensing aperture(s) 208 are located proximate to the closed end 206 of the container. The dispensing aperture(s) 208 extend through a wall of the tubular body 210 to the interior of the container 202.

The dispensing apertures 208 illustrated are square shaped, however, it can be appreciated that any number, shapes, and sizes of dispensing apertures 208 may be used. Likewise, although the container 202 shown in this example is tubular, any suitable shapes and sizes may be used. For example, the container 210 may be square shaped.

Although in the embodiments illustrated herein, the container has a non-divided interior, in other embodiments, the interior of the container can be partitioned to create separate, distinct chambers that can hold different materials or different concentrations or forms of the same material.

Also shown in FIG. 2 is a cover 216. The cover 216 may be configured to seal the open end of the container 204. The cover 216 has top and bottom surfaces. The bottom surface may be comprised of an adhesive material and/or can have an adhesive material affixed to a surface thereof to adhere the cover 216 to the container 202 for sealing. For example, in FIG. 2, the cover may be affixed to an upper surface of flange 212 to seal the open end 204. Still, other arrangements are possible. The cover may also be a label, such as a label containing product and/or advertising information.

FIGS. 3a-b illustrate a split ring 318 configured to engage an outer surface of the container 202 that may be used in accordance with an embodiment of the present invention. As can been seen in FIGS. 3a-b, the split ring 318 may be comprised of a base 320, a wall 322 extending from the base 320, and a handle 324. In the examples, since the container 202 body is tubular, the inner surface of the split ring 318 forms a semicircle to mate with an outer surface of the container 202.

It may appreciated, that the shape of the split ring 318 may be changed in accordance with the shape of the body selected for the container 202. In addition, the base 320 and wall 322 can be resilient so that the ends of the split ring 318 may be flexed inwardly and outwardly (shown in phantom in FIG. 3b) for facilitating insertion and removal of the split ring 318 from an outer surface of the tubular body 210 of the container 202.

As will be discussed below in more detail below, the split ring 318 may limit or prevent movement of the container with respect to the sleeve in the storing position (FIG. 5a).

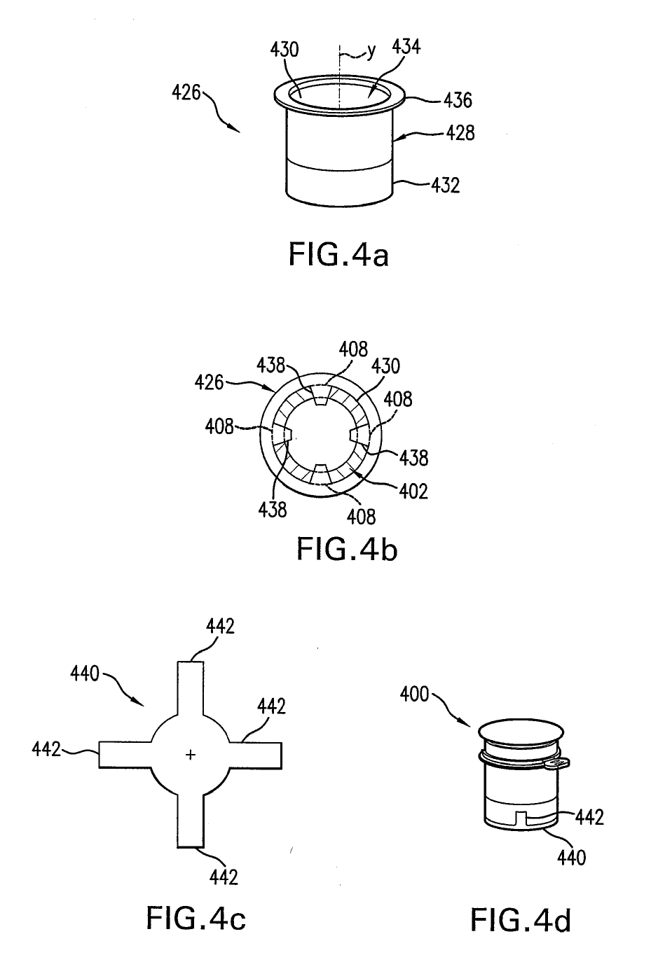

FIGS. 4a-b show side and bottom views of a sleeve 426 which may be used in accordance with an embodiment of the present invention. As best seen in FIG. 4a, the sleeve 426 has a tubular shaped body 428 with inner and outer surfaces 430, 432 defining a passageway 434. The first and second ends of the passageway 434 are open so that the container 202 can be slidably received through the passageway 432. Proximate to the first end of the sleeve is a flange 436. Similar to the flange 212 of FIG. 2, flange 436 may extend outwardly from and perpendicularly to a longitudinal axis (y) of the sleeve 426.

FIG. 4b, shows a bottom view of the sleeve 426 with the container 402 received therein. As shown in the figure, the sleeve 426 may also include one or more alignment tab(s) 438 disposed on the inner surface 430 of the sleeve 426. The alignment tab(s) 438 can extend through the dispensing aperture(s) 408 located on the container 402. One or more alignment tab(s) 438 may be used. For example, the number of alignment tab(s) 438 may be equal to the number of the dispensing aperture(s) 408 located on the container 402. Consequently, the alignment tab(s) 438 can be slidably received within the dispensing aperture(s) 408 to align the sleeve 426 and the container 402 with one another.

It can be appreciated by the embodiments of the present invention, since the alignment tab(s) 438 are interlocked within the dispensing aperture(s) 408, the distance the sleeve 426 and container 402 may move in relation to one another may be limited by the height of the dispensing aperture(s) 408. In other words, the alignment tab(s) 438 contact a lower wall of the dispensing aperture(s) 408 in the storing position (FIG. 5a) so that the lower surface of the sleeve is even or about even with the closed end of the container 402. Alternatively, in the fully dispensed position (FIG. 5b), the alignment tab(s) 438 contact the upper wall of the dispensing aperture(s) 408 so that the dispensing aperture(s) 408 extend past the lower surface of the sleeve 426.

FIG. 4c illustrates a second cover 440 and FIG. 4d illustrates the second cover 440 sealing the bottom of an apparatus 400 in the storing position as may be employed in accordance with an embodiment of the present invention. As seen in these examples, the cover 440 may have a middle portion, which may have the same cross-sectional shape as the container, and one or more arm(s) 442 which extend from the middle portion.

As seen in FIG. 4d, when the apparatus 400 is in the storing position, the second cover 440 may be positioned on the bottom of the apparatus 400 so that the middle portion may cover the closed end of the container. Further, the arm(s) 442 may be folded upwards so that they may attach to an outer surface of the sleeve. A surface of the second cover 440 may be covered with an adhesive for adhering to a target surface. The second cover 440 may also be a label, such as a label containing product and/or advertising information.

The arm(s) 442 may be arranged so that they are aligned with the dispensing aperture(s) of the container. The arm(s) 442 may provide additional sealing means to prevent or limit the material from exiting from the interior of the container during, for example, shipping of the apparatus 400.

Referring to FIG. 5a-b, a bottom cap can be used in addition to or as an alternate to second cover 440 to sealing the bottom of an apparatus 400 in the storing position. Although the bottom cap may have any suitable height that allows it to seal the bottom of apparatus 400, in certain embodiments, when bottom cap 10 is fitted over apparatus 400 as shown in FIG. 5b, bottom cap 10 has a height that is between about 25% to 80% of the height of the sleeve. In a preferred embodiment, bottom cap 10 has a height that is about 75% of the height of the sleeve.

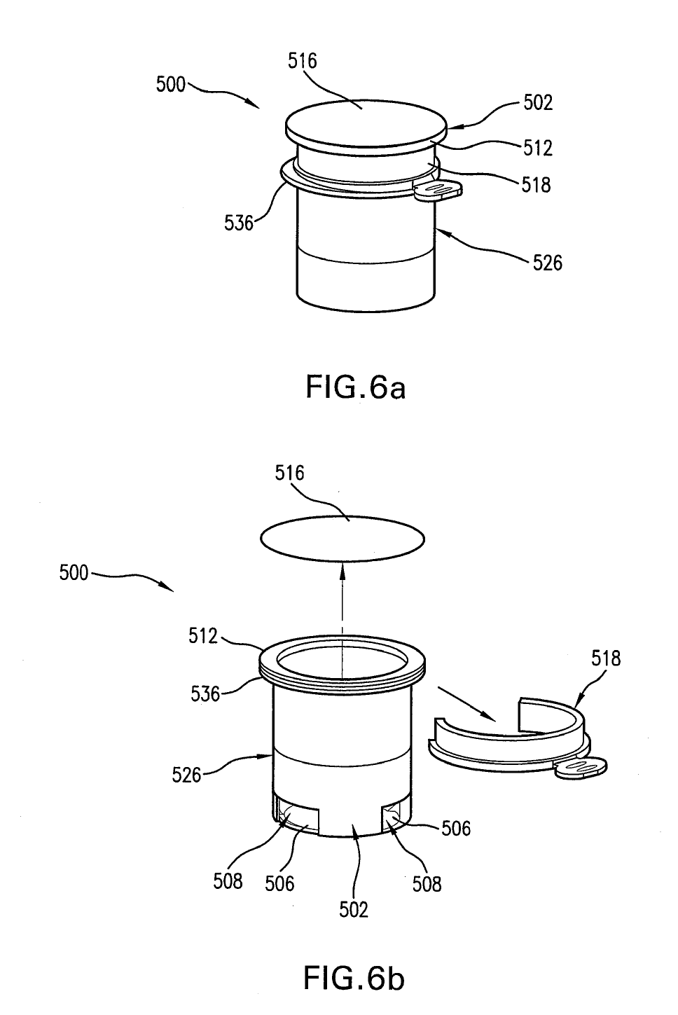

As stated herein above, FIG. 6a shows the apparatus 500 in the storing position and FIG. 6b shows the apparatus in the dispensing position. In the storing position, the container 502 can be inserted within the sleeve 526 so that sleeve 526 covers the dispensing apertures 508. In addition, the alignment tabs of the sleeve 526 may be inserted within the dispensing apertures (shown in FIG. 4b) so that the tabs are positioned against the lower wall of the dispensing aperture adjacent to the closed end of the container 502.

Also as seen in FIG. 6a, in the storing position, a space may be formed between the flange 512 of the container and the flange 536 of the sleeve. This space or gap formed between the flanges 512, 536 may be configured to receive the split ring 518. The split ring 518 may then be inserted into this space or gap to prevent or limit movement of the container 502 and sleeve 526 with respect to one another in the storing position. For example, a frictional fit between the container 502 and sleeve 526, coupled with the split ring, can prevent or limit movement of the these components during shipping, therefore prevent material from exiting the interior of the container 502.

Prior to insertion or following insertion of the split ring 518, the container 502 may be filled with a material or materials that are desired to be reconstituted (and which are described in more detail below).

Referring again to FIG. 6a, once the container 502 is filled with material, the cover 516 may be positioned on the container to seal the open end thereof. Consequently, in the storing position, the cover 516 may cover the open end of the container 502 and the sleeve 526 may cover the dispensing aperture(s) 508 to limit and/or prevent the material or materials from exiting the interior of the container 502. Likewise, a second cover may be used as discussed herein above with respect to FIGS. 4c-d to provide additional sealing.

FIG. 6b shows the apparatus 500 in the dispensing position. In this example, the cover 516 and split ring 518 are removed. It can be appreciated by embodiments of the invention that the cover 516 does not need to be removed and may be left on to prevent material from exiting the open end if the apparatus is, for example, agitated to release material.

For dispensing, a user may position the apparatus 500 in a desired location. For example in systems in accordance with embodiments of the present invention described herein below, the apparatus 500 may be used in conjunction with a reservoir for holding a substance in which the material in the apparatus is reconstituted. Once in the desired position, the container may be moved from the storing position (FIG. 6a) to the dispensing position (FIG. 6b). As this movement occurs, in this example, the flange 512 on the container 502 moves toward the flange 536 on the sleeve 526.

Since the container 502 can be longer than the sleeve 526, as the container 502 is moved, the closed end 506, including the dispensing aperture(s) 508, moves past the lower surface of the sleeve 526 to uncover the dispensing aperture(s) 508. At this time, the material can exit from the interior of the container 502 by, for example, gravity and/or agitation of the apparatus 500.

As stated herein above, in the fully dispensed position, the alignment tabs (FIG. 4b) of the sleeve 526 contact the upper wall of the dispensing aperture.

In some examples, surfaces of the closed end 506 may be sloped to facilitate dispensing of the material. In other examples, the apparatus 502 may be agitated to facilitate dispensing of the materials. In still other examples, fluid (e.g., liquid and/or air) may be provided to the interior of the container 502 to facilitate dispensing and/or mixing of the material.

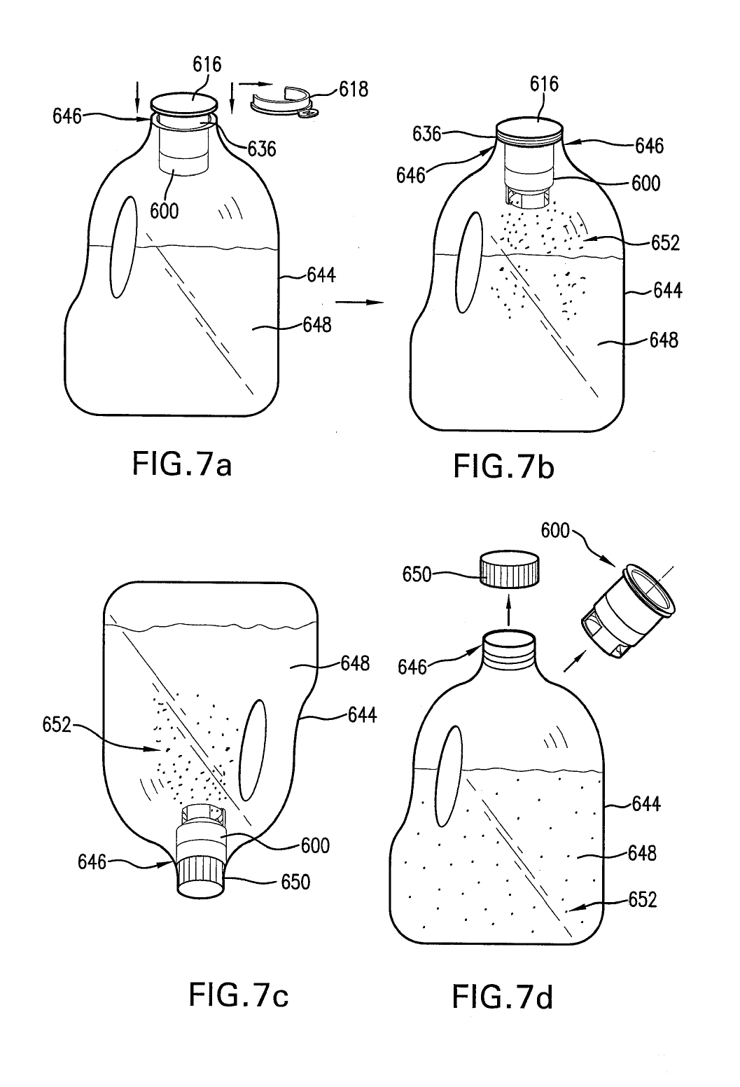

Turning to FIGS. 7a-d, systems in accordance with embodiments of the present invention for storing, dispensing, and reconstituting materials will now be described.

In the examples, a reservoir 644 is shown in the form of a jug and is shown having an opening 646 and an interior containing a substance 648. Of course, other sizes and configurations of a reservoir can also be used such as gallon sized drums, bottles, ampoules, needles, or other configurations. In the example, the substance is a liquid but other substances such as solids, and/or gases can be used. In certain embodiments the substance, when a solid, is in powdered form. Non-limiting examples of a liquid include distilled water, however, other suitable fluids may be used. The reservoir 644 can be, but need not be, filled to capacity.

As seen in FIG. 7a, a user may position the apparatus 600 in the opening 646 of the reservoir 644 via passive or active methods including threading the apparatus into the reservoir (in which case opening 646 is a complementary threaded opening). In other embodiments, the apparatus is integrally associated with the reservoir (i.e. by being pre-formed or otherwise molded with the reservoir such that the apparatus need not be inserted into the reservoir but rather is already positioned in the opening of the reservoir) Split ring 618 may then be removed. It can be appreciated that the split ring 618 may also be removed prior to inserting the apparatus 600 in the opening 646 of the reservoir 644. In addition, the cover 616 may be removed if desired, but need not be, at any time during the dispensing process.

The flange 636 of the sleeve may be sized to fit over the reservoir opening 646. Consequently, upon insertion of the apparatus 600 into the opening 646, the flange 636 rests on top of the opening 646 while other portions of the sleeve extend into the reservoir 644. Since the container is also positioned within the sleeve, portions of the container also extend into the reservoir 644.

As seen in FIG. 7b, in order to dispense the material(s) 652, the user moves the container towards the sleeve, such as by placing pressure on a top surface of the flange 636 of the container or twisting the container or using another mechanical method. As the container moves toward the sleeve, since the body of the container is longer than that of the sleeve, the lower portion of the container, including the dispensing apertures, move past a lower surface of the sleeve. As the container moves past the sleeve, the dispensing apertures are uncovered by the sleeve and thus dispense material 652 into the interior of the reservoir 644, for example, by gravity and/or agitation.

Upon contact with the material 652, the fluid 648 may dissolve the material 652 thereby creating a solution.

As seen in FIG. 7c, a reservoir cap 650 may also be used. For example, once the apparatus 600 is placed within the opening 646 of the reservoir 644, the reservoir cap 650 may be used to seal the apparatus 600 between the reservoir 644 and the reservoir cap 650. The reservoir 644 and reservoir cap 650 may be threabably engageable with one another to facilitate sealing of the apparatus 600 therebetween. Once the apparatus 600 is sealed, as seen in FIG. 7c, the reservoir may inverted, as well as agitated (e.g. shaking reservoir), to further mix material 652 with the fluid 648.

FIG. 7d shows the material 652 dissolved within the liquid 648 of the reservoir 644. The reservoir cap 650 may be removed and the apparatus 600 removed from the reservoir opening 646. Accordingly, the reservoir cap 650 may be placed back on the reservoir 644 and the solution stored for subsequent and/or immediate use.

The apparatuses of the present invention may be fabricated from any suitable material. Non-limiting examples include a polymer or metal. In certain embodiments, the material is a plastic. In certain embodiments, the material is non-eluting, biocompatible and/or dissolvable/biodegradable. Regarding the latter feature, the material could be any suitable dissolvable material such as a gel.

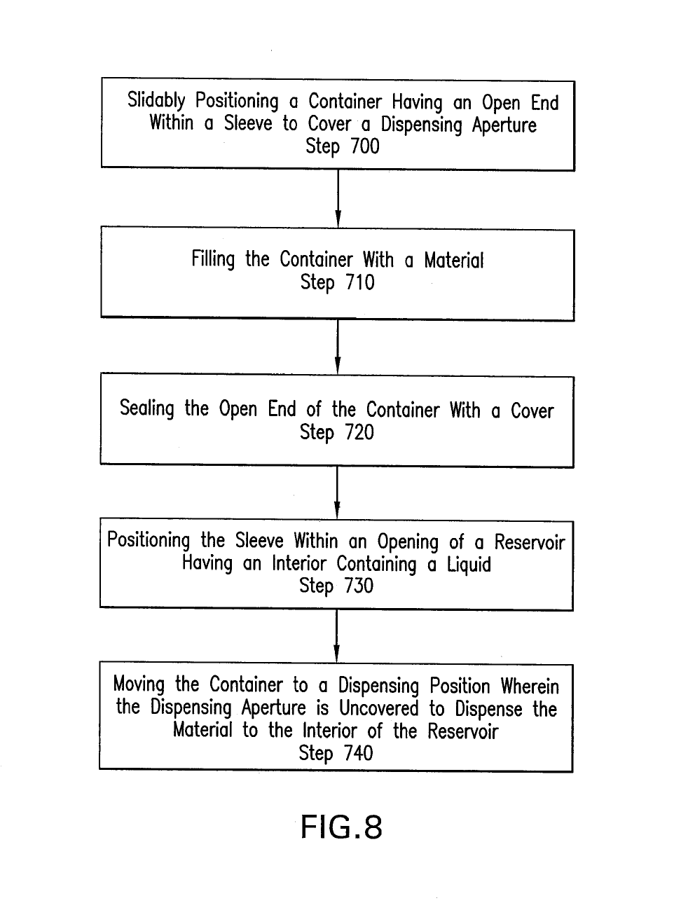

FIG. 8 shows a flow chart including method steps that may be employed with embodiments of the present invention for storing and dispensing a material from an apparatus. In the example of FIG. 8, step 700 may include slidably positioning a container having an open end within a sleeve to cover a dispensing aperture. Step 710 may include filling the container with a material. Step 720 may include sealing the open end of the container with a cover. Step 730 can include positioning the sleeve within an opening of a reservoir having an interior containing a liquid. Step 740 may include moving the container to a dispensing position wherein the dispensing aperture is uncovered to dispense the material to the interior of the reservoir. Additional steps can include mixing the material with the substance in the reservoir. In other embodiments, materials may be mixed in the apparatus in addition to or as an alternative to mixing the materials in a reservoir.

In other embodiments the sequence of steps may be reordered and steps may be added or removed. The steps may also be modified.

As mentioned above, a container of the present invention may be filled with a material that is desired to be reconstituted. The material could be in any suitable form including a gas, liquid, and/or solid. The material could also be in one or two different forms. For example, the material may be in a powdered form, a liquid/powder form, a powder/powder form or a liquid/liquid form. The material could also be in the form a capsule or tablet with an outer film layer. When the material is exposed to a substance in a reservoir, such as a liquid, the film would dissolve releasing the constituents of the material through the dispensing apertures of the container.

In certain embodiments, the material is an electrolyte formulation. The term “electrolyte formulation” as used herein may include a source of salt, which may be dissolved in distilled water to form an electrolyte saline solution which can subsequently undergo electrolysis to form an electrolyzed saline solution. Such an electrolyzed saline solution can include a mixture of active species, including predominantly hypohalous acid (HOX), wherein X is a halide. A halide is an anion such as fluoride (F⁻), chloride (Cl⁻) bromide (Br⁻), iodide (I⁻) or astatide (At⁺). Therefore, the salt source can be a halide salt, including, for example, sodium chloride, sodium bromide, sodium fluoride, sodium iodide, potassium chloride, potassium bromide, potassium fluoride, and/or potassium iodide.

An electrolyte formulation which may be useful for embodiments of the present invention may also contain a buffer. A buffer may be used to affect the pH of the electrolyte formulation when it is reconstituted with water as described below to form an electrolyzed solution. The pH of the reconstituted electrolyzed solution can be, in some examples, about 4 to about 8 and more preferably about 5 to about 7. Buffers which may be useful in achieving the desired pH are known in the art, including, for example carbonates and/or hydroxides. Specific exemplary buffers include, but are not limited to, sodium carbonate, potassium carbonate, sodium hydroxide, and/or sodium bicarbonate.

An electrolyte formulation or other material stored in a container of the present invention can also include one or more colorants. The colorant may impart color to one or more of the electrolyte formulation, the saline solution, and/or the electrolytic saline solution. Colorants that can be used in accordance with embodiments of the present invention include synthetic or natural colorants. Non-limiting examples of suitable colorants include dyes such as, for example, vegetable dyes, organic dyes, and inorganic dyes. Other examples of colorants include pigments, including, for example, mineral pigments.

An electrolyte formulation or other material stored in a container of the present invention can also include a therapeutic agent. Non-limiting examples of therapeutic agents include a antimicrobial agent, an antifungal agent, an anti-inflammatory agent, an anti-itch agent, an antiseptic, an analgesic, a vitamin, an amino acid, a herbal extract, a growth factor, an antioxidant, a cosmetic agent, a skin conditioner, and any combination thereof. The therapeutic agent can also be in the form of a nanoparticle that can chemically change during electrolysis to have a function different than prior to electrolysis. The electrolyte formulation can also include other nanoparticles.

An electrolyte formulation or other material stored in a container of the present invention can also include one or more surfactants. Surfactants that can be used in accordance with the present invention include anionic surfactants, cationic surfactants, nonionic surfactants, amphoteric surfactants, ampholytic surfactants, and zwitterionic surfactants.

In a preferred embodiment, an electrolyte formulation useful for the present invention is in powder form. By preparing the electrolyte as a dry powder formulation, the electrolyte formulation may be transported more easily and can have a longer shelf life than, for example, a solution reconstituted off-site.

An electrolytic formulation used to form an electrolyzed saline solution can have many different uses. For example, an electrolyzed saline solution can be used as a disinfectant or decontaminate. The disinfectant or decontaminate can be used to remove, kill or otherwise inactivate harmful pathogens from a surface. Such surfaces include, for example, surfaces that come into human contact or are used to produce water, food or other substances for human consumption. For example, a disinfectant can be used to remove biofilm matrices and inactivate pathogens from surfaces in medical and non-medical environments. Furthermore, disinfecting solutions may be used in the dental field. In the dental field, the quality of the water that flows through dental unit water lines may affect the health of patients during dental procedures and that of staff when they inhale aerosol mist from dental hand tools (which are connected to the water lines). Consequently, disinfecting solutions may be used to facilitate the removal of biofilm in dental unit water lines and to maintain the water quality of dental lines over time.

In still other examples, disinfecting solutions may be used for food preservation, general disinfection in medical applications, water treatment, and general agricultural and petrochemical uses. The disinfectants and decontaminants can be electrolyzed saline solutions or other forms of disinfectants or decontaminants.

Of course it is understood that the apparatuses of the present invention can be used to store one or more materials other than electrolyte formulations. For example, an apparatus of the present invention can be used to store ingredients for making beverages such as, for example, energy or fruit drinks, carbonated beverages, alcoholic beverages and/or water. Non-limiting examples of ingredients include additives, such as, for example, caffeine, carbon dioxide, nutrients, flavoring, sugar, growth factors, vitamins (including, for example, vitamin A, D, E, K, and C), antioxidants, minerals, fermentation additives (for alcoholic beverages such as wine) and/or electrolytes. The additives can be natural and/or synthetic and can be in concentrated or un-concentrated form.

Other non-limiting exemplary materials that can be used in apparatuses of the present invention include materials that have medicinal use. Non-limiting examples of medicinal uses include creating a solution for oral, gastrointestinal, topical, or nasal uses, for example. Regarding oral uses, a material can be ingredients used to create, for example, a mouth wash, an oral rinse, a plaque barrier, and/or a treatment for caries disease. Regarding gastrointestinal uses, a material stored in an apparatus of the present invention can be used to make a bowel cleansing solution for a colonoscopy. Regarding topical uses, a material stored in an apparatus of the present invention can be used to make a skin care product, for example, for treating skin diseases or conditions. Non-limiting examples of skin conditions include diabetic, venous and/or pressure sores, skin abrasions, burns or other types of wounds, including open wounds. Other medicinal uses include using a material stored in an apparatus of the present invention to form an antimicrobial solution. A material stored in an apparatus of the present invention can also be a pre-mixed powder or liquid to be used in pharmacological compounding (such as, for example, sulfamylon which can be released into a compounding bottle for easier preparation or other materials that can be reconstituted).

Other non-limiting exemplary materials that can be used in apparatuses of the present invention include materials that are used to treat water. For example, a material stored in an apparatus of the present invention can be a carbon absorbent or other material used to decontaminate low quality water.

The apparatuses can be used to store materials used for other purposes as well. For example, the material can be a substance used for cosmetic purposes. For example, an apparatus of the present invention can be used in the hair coloring industry to mix components to activate the coloring agent. For example, the activator could be in the apparatus and could contain the colorant and a chemical activator. When released into a receptacle and mixed, the chemical activator to could activate the colorant. Of course, the above-described uses are only exemplary and an apparatus of the present invention can be used for other purposes where it is desired to mix any two or more components together.

The present invention also provides kits including electrolytic formulations and other formulations and materials as described above and apparatuses and systems as described above as well as methods of using such kits, systems and apparatuses as described above

The examples described herein are merely illustrative, as numerous other embodiments may be implemented without departing from the spirit and scope of the exemplary embodiments of the present invention. For example, although the apparatuses of the present invention are described with respect to an electrolyte formulation, the apparatuses could be used for other types of formulations or materials that are to be reconstituted or otherwise mixed with another material. Moreover, while certain features of the invention may be shown on only certain embodiments or configurations, these features may be exchanged, added, and removed from and between the various embodiments or configurations while remaining within the scope of the invention.

Likewise, methods described and disclosed may also be performed in various sequences, with some or all of the disclosed steps being performed in a different order than described while still remaining within the spirit and scope of the present invention.

What is claimed is:

1. An apparatus for storing and dispensing a material, comprising:
a container to store the material having an open end and a dispensing aperture;
a cover configured to seal the open end of the container; and
a sleeve including a passageway to slidably receive the container;
the container moveable within the sleeve between a storing position wherein the dispensing aperture is covered and a dispensing position wherein the dispensing aperture is uncovered to dispense the material.

2. The apparatus of claim 1, further comprising a split ring located between the container and the sleeve.

3. The apparatus of claim 2, wherein the split ring is located between a first flange located on the container and a second flange located on the sleeve.

4. The apparatus of claim 3, wherein the split ring limits movement of the container in the storing position.

5. The apparatus of claim 1, wherein the container has a closed end and the dispensing aperture is located proximate to the closed end.

6. The apparatus of claim 1, wherein the dispensing aperture is a plurality of apertures.

7. The apparatus of claim 1, wherein the cover is removable.

8. The apparatus of claim 1, further comprising a second cover configured to seal the container including the dispensing aperture and the sleeve in the storing position.

9. The apparatus of claim 1, further comprising a bottom cap configured to seal the sleeve in a storing position, the bottom cap having a height that is between about 25% to 80% of the height of the sleeve.

10. The apparatus of claim 1, wherein the sleeve has a tab located on an inner surface and the dispensing aperture is configured to slidably receive the tab.

11. A system for storing and dispensing a material, comprising:
a container to store the material having an open end and a dispensing aperture;
a cover configured to seal the open end of the container;
a sleeve including a passageway to slidably receive the container, the container moveable within the sleeve between a storing position wherein the dispensing aperture is covered and a dispensing position wherein the dispensing aperture is uncovered to dispense the material; and
a reservoir having an opening,
wherein the sleeve is configured for positioning on the opening so that when the container is moved to the dispensing position the material is dispensed into the reservoir to form a reconstituted material.

12. The system of claim 11, wherein the sleeve has a flange for mounting on the reservoir opening.

13. The system of claim 11, wherein the container has a closed end and the dispensing aperture is located proximate to the closed end.

14. The system of claim 11, wherein the dispensing aperture is a plurality of apertures.

15. The system of claim 11, further comprising a cap to seal the liquid reservoir when the container is in the dispensing position.

16. The system of claim 15, wherein the cap is threadably engageable with the opening of the liquid reservoir.

17. The system of claim 11, wherein a slip ring is positionable between a flange of the container and a flange of the sleeve to limit movement of the container in the storing position.

18. The system of claim 11, wherein the cover is removable.

19. The system of claim 11, wherein the sleeve has an alignment tab located on an inner surface and the dispensing aperture is configured to slidably receive the alignment tab.

20. A kit comprising the apparatus of claim 1, and further comprising a material comprising an electrolyte formulation.

21. The kit of claim 20, wherein the electrolyte formulation is in a powder form.

22. The kit of claim 20, wherein the electrolyte formulation is in a solid or liquid form.

23. The kit of claim 20, wherein the electrolyte formulation comprises a salt.

24. The kit of claim 23, wherein the salt is a halide salt.

25. The kit of claim 20, wherein the electrolyte formulation further comprises a colorant.

26. The kit of claim 20, wherein the electrolyte formulation further comprises a surfactant.

27. The kit of claim 20, wherein the electrolyte formulation further comprises a therapeutic agent.

28. A kit comprising the apparatus of claim 1, and further comprising a material that is capable of being reconstituted into a disinfectant or decontaminant to remove, inhibit, kill or otherwise inactivate a pathogen.

29. A kit comprising the apparatus of claim 1, and further comprising an ingredient for incorporating into a beverage.

30. The kit of claim 28, wherein the ingredient is caffeine, carbon dioxide, a nutrient, a flavoring, a growth factor, a vitamin, an antioxidant, a mineral and/or an electrolyte.

31. The kit of claim 29, wherein the beverage is water, a fruit drink, an energy drink, a carbonated beverage, or an alcoholic beverage.

32. The kit of claim 31, wherein the beverage is an alcoholic beverage and the ingredient is a fermentation additive.

33. The kit of claim 30, wherein the vitamin is vitamin A, D, E, K and/or C.

34. The kit of claim 29, wherein the ingredient is a natural ingredient.

35. The kit of claim 29, wherein the ingredient is a synthetic ingredient.

36. The kit of claim 29, wherein the ingredient is in concentrated form.

37. A kit comprising the apparatus of claim 1, and further comprising a material comprising a therapeutic agent.

38. A method of using the system of claim 11 comprising:
providing a material in the container;
providing a substance in the reservoir;
positioning the sleeve on the opening of the reservoir;
moving the container to a dispensing position, wherein the dispensing aperture is uncovered to dispense the material to the interior of the reservoir; and
mixing the material in the container with the substance in the reservoir to form a reconstituted material.

39. The method of claim 38, wherein the substance in the reservoir is a liquid, a gas or a solid.

40. The method of claim 38, wherein the material in the container is a liquid, gas or a solid.

41. The method of claim 38, wherein the reconstituted material is an agent to remove, inhibit, kill or otherwise inactivate a pathogen.

42. The method of claim 38, wherein the reconstituted material is an electrolyte saline solution.

43. The method of claim 42, wherein the electrolyte saline solution is used to form an electrolyzed saline solution.

44. The method of claim 43, wherein the electrolyzed saline solution comprises a hypohalous acid.

45. The method of claim 43, further comprising disinfecting or decontaminating a surface by applying the electrolyzed saline solution to the surface.

46. The method of claim 43, further comprising passing the electrolyzed saline solution through a water line.

47. The method of claim 43, further comprising using the electrolyzed saline solution to remove biofilm from a dental unit water line.

48. The method of claim 43, further comprising rinsing a food product with the electrolyzed saline solution.

49. The method of claim 38, wherein the reconstituted material is a solution suitable for use in the gastrointestinal tract.

50. The method of claim 49, further comprising irrigating the colon with the solution.

51. The method of claim 49, wherein the reconstituted material is a material suitable for topical use.

52. The method of claim 51, further comprising applying the reconstituted material to the skin to treat a skin condition.

53. The method of claim 52, wherein the skin condition is a wound, burn, abrasion, and/or a sore.

54. The method of claim 53, wherein the sore is a diabetic, venous, or pressure sore.

55. The method of claim 38, wherein the reconstituted material is a solution suitable for oral use.

56. The method of claim 55, wherein the reconstituted material is an oral rinse, a mouth wash, a plaque barrier and or a therapeutic agent to treat caries disease.

57. The method of claim 38, wherein the reconstituted material is an antimicrobial agent.

58. The method of claim 38, wherein the reconstituted material is a carbon absorbent.

59. The method of claim 58, further comprising applying the carbon absorbent to water.

60. The method of claim 38, wherein the reconstituted material is suitable for cosmetic purposes.

61. The method of claim 60, wherein the material in the container is an agent to activate a coloring agent and the substance in the reservoir is a coloring agent.

62. A method of using the system of claim 11 comprising:
providing at least one material in the container;
providing at least one substance in the reservoir;
positioning the sleeve on the opening of the reservoir;
moving the container to a dispensing position, wherein the dispensing aperture is uncovered to dispense the material to the interior of the reservoir; and
mixing the at least one material in the container with the at least one substance in the reservoir to form a reconstituted material.

63. The method of claim 62, wherein the at least one material comprises one or more different materials.

64. The method of claim 62, wherein the at least one substance comprises one or more substances.
